(19) United States
(12) Patent Application Publication
(10) Pub. No.: US 2024/0058314 A1
(43) Pub. Date: Feb. 22, 2024

(54) COMPOSITIONS COMPRISING ANATABINE AND USES THEREOF

(71) Applicant: Philip Morris Products S.A., Neuchatel (CH)

(72) Inventors: Julia HOENG, Corcelles (CH); Carine POUSSIN, Evian-les-Bains (FR); Dimitrios MESSINIS, Neuchatel (CH)

(73) Assignee: Philip Morris Products S.A., Neuchatel (CH)

(21) Appl. No.: 18/260,293

(22) PCT Filed: Jan. 7, 2022
(86) PCT No.: PCT/EP2022/050293

§ 371 Date: Jul. 3, 2023

(30) Foreign Application Priority Data
Jan. 7, 2021 (EP) 21386001.8

(51) Int. Cl. A61K 31/444

(52) U.S. Cl. CPC A61K 31/444

(57) ABSTRACT

A composition including anatabine or a salt thereof is provided for treating a nuclear factor erythroid 2-related factor 2 (NRF2)-regulated disorder.) A composition including anatabine or a salt thereof is also provided for prophylactic treatment of a subject requiring NRF2 activation.

### FIELD OF INVENTION

The present invention relates to a composition comprising anatabine or a salt thereof, in particular a composition comprising anatabine or a salt thereof for use in the treatment of a nuclear factor erythroid 2-related factor 2 (NRF2)-regulated disorder.

### BACKGROUND

Nuclear factor erythroid 2-related factor 2 (NRF2) is a central regulator of redox, metabolic and protein homeostasis that intersects with many other signalling cascades. NRF2 is also known as nuclear factor erythroid-derived 2-like 2 (NFE2L2) which is a transcription factor that is encoded by the NFE2L2 gene in humans. Structurally, NRF2 is a basic leucine zipper (bZIP) protein which participates in regulating the expression of a plurality of human genes that enhance the antioxidant response and represents a promising pharmacological target especially for chronic diseases characterized by low-grade oxidative stress and inflammation.

Some phytochemicals have been shown to exert their anti-inflammatory effect concomitantly to an anti-oxidant effect through the activation of NRF2. Known herbal supplements on the market include supplements which are based on curcumin (turmeric) in combination with other herbs such as Protandim®, NRF2 Activator (Xymogen®), NRF2 Optimizer (Nuley), NRF2 Antiox (Progressive Nutracare), and Ultimate Protector™.

An abundance of molecules have been described as NRF2 inducers but only a few dozen are in clinical development. However, only four molecules—dimethyl fumarate (DMF), bardoxolone methyl (BARD-Me), oltipraz, and sulforaphane (SFN)—have appeared substantively in peer-reviewed literature, especially where measures of biomarkers have been a component of the clinical protocols.

Most of these substances are electrophilic and appear to activate NRF2 through the modification of cysteines, or binding to the Kelch domain of KEAP1. Of these clinically tested NRF2 activators, only Tecfidera® (DMF) has been approved for the treatment of Multiple Sclerosis by the U.S. Food and Drug Administration in 2013 and the European Medicines Agency in 2014.

Thus, there is a need for an improved NRF2 activator, an improved composition for NRF2 activation and its use in the treatment of NRF2-regulated disorders.

### SUMMARY OF INVENTION

Anatabine is a minor nucleophile alkaloid found in plants of the family Solanaceae. Anatabine is also known as 1,2,3,6-Tetrahydro-2,3′-bipyridine and as 3-(1,2,3,6-tetrahydropyridin-2-yl) pyridine and has the following Formula I:

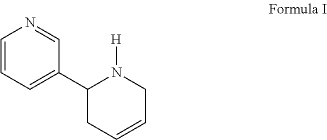 [SMILES: C1=CCC(c2cccnc2)NC1]

According to a first aspect of the invention, there is provided a composition comprising anatabine or a salt thereof for use in the treatment of a nuclear factor erythroid 2-related factor 2 (NRF2)-regulated disorder.

Anatabine has been found to activate NRF2 and thus anatabine or a salt thereof may be used in the treatment of a NRF2-regulated disorder. This is particularly surprising because other tobacco alkaloids (e.g., nornicotine, nicotine, cotinine, anabasine) do not substantially affect NRF2 activity. It is also particularly surprising that anatabine, which is nucleophilic, activates NRF2 activity because most known NRF2 activators are electrophilic. Advantageously, anatabine is better-tolerated than known NRF2 activators (e.g., sulforaphane) making anatabine particularly suited for use in the treatment of a NRF2-regulated disorder.

The anatabine salt may be a pharmaceutically acceptable salt or a food grade salt. The salt may be selected from one of l-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid (L), aspartic acid (L), benzenesulfonic acid, benzoic acid, camphoric acid (+), camphor-10-sulfonic acid (+), capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid (D), gluconic acid (D), glucuronic acid (D), glutamic acid, glutaric acid, glycerophosphoric acid, glycolic acid, hippuric acid, hydrobromic acid, hydrochloric acid, isobutyric acid, lactic acid (DL), lactobionic acid, lauric acid, maleic acid, malic acid (−L), malonic acid, mandelic acid (DL), methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, pyroglutamic acid (−L), salicylic acid, sebacic acid, stearic acid, succinic acid, sulfuric acid, tartaric acid (+L), thiocyanic acid, toluenesulfonic acid (p), and undecylenic acid. Preferably, the salt may be selected from one of adipate, tartrate, citrate, L-aspartate, camphor-10-sulphonate, cinnamate, cyclamate, fumarate, D-gluconate, L-glutamate, glutarate, L-(+)-lactate, (±)-DL-lactate, maleate, malate, oxalate, galactarate, glucoheptonate, hippurate, malonate, (±)-DL-mandelate, nicotinate, salicylate, and/or succinate. Alternatively, the anatabine may be the free base.

The expressions “disorder” and “disease” may be used interchangeably. The treatment of the NRF2-regulated disorder may comprise activation of NRF2. The terms “activation of NRF2” or “activates NRF2” are understood to include an increase in NRF2 activity and/or NRF2 translocation to the cell nucleus. The treatment may comprise curative, prophylactic or restorative treatment of a NRF2-regulated disorder. The treatment may comprise promotion of an anti-oxidative response. The treatment may comprise anti-inflammatory effects. The disorder may be treated or prevented by anatabine treatment (curative or prophylactic) through an increase of NRF2 activity. The treatment may comprise the improvement of the condition of a subject where the condition may be NRF2-regulated.

Optionally, the composition according to the invention may be for use in the treatment of a disorder which is NF-κB independent, i.e the composition according to the invention may be for use in the treatment of a disorder which is not NF-κB-regulated. The NRF2-regulated disorder may be a nuclear factor kappa-light-chain-enhancer of activated B cells (NF-κB) independent disorder.

The composition for use according to the invention may regulate the inflammatory response. The treatment of a NRF2-regulated disorder may comprise a step of administering anatabine or a salt thereof to regulate, e.g. reduce, the inflammatory response. The invention may also provide the use of a composition comprising anatabine or a salt thereof as an inflammatory response regulator. The invention may also provide a method for reducing inflammatory response, the method comprising administering to a subject an effective dose of anatabine or a salt thereof.

The composition for use according to the invention may regulate oxidative stress. The treatment of a NRF2-regulated disorder may comprise a step of administering anatabine or a salt thereof to regulate, e.g. reduce, the oxidative stress. The invention may also provide use of a composition comprising anatabine or a salt thereof as an oxidative stress regulator. The invention may also provide a method for reducing oxidative stress, the method comprising administering to a subject an effective dose of anatabine or a salt thereof.

The composition for use according to the invention may be for use in the treatment of a subject. The subject may be a subject at risk of having and/or developing a NRF2-regulated disorder. The subject may be a subject requiring NRF2 activation.

The NRF2-regulated disorder may be selected from one or more of a Heme Oxygenase 1 (HMOX1)-regulated disorder, a Glutamate-cysteine ligase (GCLM)-regulated disorder, a NAD(P)H Quinone Dehydrogenase 1 (NQO1)-regulated disorder, and/or Thioredoxin Reductase 1 (TXNRD1)-regulated disorder. Advantageously, anatabine or a salt thereof activates NRF2 which controls HMOX1, GCLM, NQO1 and/or NQO1.

The HMOX1-regulated disorder may be Heme Oxygenase 1 deficiency or chronic obstructive pulmonary disease (COPD). Among its related pathways are photodynamic therapy-induced NFE2L2 (NRF2) survival signalling and validated transcriptional targets of AP1 family members Fra1 and Fra2. Gene Ontology (GO) annotations related to this gene include protein homo-dimerisation activity and oxidoreductase activity. An important paralog of this gene is HMOX2.

The HMOX1-regulated disorder may be a BLVRB Flavin reductase (NADPH)-regulated disorder, a Glutamate-cysteine ligase catalytic subunit (GCLC)-regulated disorder, a Catalase (CAT)-regulated disorder, a Speckle targeted PIP5K1A-regulated poly(A) polymerase (TUT1)-regulated disorder, a RAC-alpha serine/threonine-protein kinase (AKT1)-regulated disorder, a NADPH-cytochrome P450 reductase (POR)-regulated disorder.

The GCLM-regulated disorder may be haemolytic anaemia, myocardial infarction, Parkinson disease and ischemic heart disease. The NQO1-regulated disorder may be tardive dyskinesia (TD), cancer, Alzheimer's disease (AD). The TXNRD1-regulated disorder may be diphtheria and exencephaly.

The NRF2-regulated disorder may be a disorder regulated by any of the following genes:

Activating Transcription Factor 3 (ATF3), Chromosome 16 Open Reading Frame 72 (C16orf72), CTD Small Phosphatase 1 (CTDSP1), Cytochrome P450 Family 1 Subfamily B Member 1(CYP1B1), DnaJ Heat Shock Protein Family (Hsp40) Member B4 (DNAJB4), Eukaryotic Elongation Factor 2 Kinase (EEF2K), EP300 Interacting Inhibitor Of Differentiation 3 (EID3), Family With Sequence Similarity 53 Member C (FAM53C), F-Box Protein 30 (FBXO30), G0/G1 Switch 2 (G0S2), Glutamate-Cysteine Ligase Modifier Subunit (GCLM), Heme Oxygenase 1 (HMOX1), Heat Shock Protein Family A (Hsp70) Member 1 Like (HSPA1L), MAF BZIP Transcription Factor F (MAFF), MIR22 Host Gene (MIR22HG), Proteasome Assembly Chaperone 4 (PSMG4), SERTA Domain Containing 1 (SERTAD1), Solute Carrier Family 7 Member 11 (SLC7A11), Transcription Factor Binding To IGHM Enhancer 3 (TFE3), TNF Superfamily Member 9 (TNFSF9), Thioredoxin Reductase 1 (TXNRD1), Zinc Finger And BTB Domain Containing 21 (ZBTB21), Zinc Finger AN1-Type Containing 2A (ZFAND2A).

Preferably, the NRF2-regulated disorder may be a disorder regulated by any of the following genes HMOX1, TXNRD1, GCLM, SLC7A11, FBXO30, ATF3, MAFF, EEF2K, TFE3, MIR22HG, DNAJB4.

The NRF2-regulated disorder may be a disorder or disease selected from one or more of the following: a neurodegenerative disease, an inflammatory disease, an auto-immune disease, a cardiovascular disease, a respiratory disease, a digestive system disease, a neoplasm, and/or a glucose metabolism disease. The neurodegenerative disease may be amyotrophic lateral sclerosis, Parkinson's disease, Alzheimer's disease, Huntington's disease, and prion diseases. The inflammatory disease may be an allergy, asthma, autoimmune disease, coeliac disease, glomerulonephritis, hepatitis, inflammatory bowel disease, preperfusion injury and transplant rejection. The auto-immune disease may be Type 1 diabetes, rheumatoid arthritis, psoriasis/psoriatic arthritis, multiple sclerosis, systemic lupus erythematosus, inflammatory bowel disease, Addison's disease, Graves' disease, Sjögren's syndrome, Hashimoto's thyroiditis, Myasthenia gravis, autoimmune vasculitis, pernicious anaemia, and/or celiac disease. The cardiovascular disease may be coronary heart disease, e.g. angina, heart attacks, heart failure, strokes, transient ischaemic attacks, peripheral arterial disease, aortic disease. The respiratory disease may be asthma, chronic obstructive pulmonary disease (COPD), pulmonary fibrosis, pneumonia, and lung cancer. The digestive system disease may be constipation, irritable bowel syndrome, haemorrhoids, anal fissures, perianal abscesses, anal fistulas, perianal infections, diverticular diseases, colitis, colon polyps and cancer. The neoplasm may be abnormal growth of cells, i.e. a tumour. The glucose metabolism disease may be diabetes, galactosemia, Hunter syndrome, Hurler syndrome, mucopolysaccharidoses, Pompe disease, and/or Type I glycogen storage disease.

The NRF2-regulated disorder may be autism spectrum disorder (ASD). ASD may be selected from one or more of the following: autistic disorder, pervasive developmental disorder and/or Asperger syndrome. Advantageously, anatabine is suitable for use in the treatment of a NRF2-regulated disorder, for example, wherein the NRF2-regulated disorder is autism spectrum disorder (ASD). Numerous studies in ASD patients have demonstrated NRF2-regulated alterations in inflammation/immune, oxidative stress, and mitochondrial dysfunction.

The anatabine or salt thereof may be a racemic mixture. The anatabine or salt thereof may be the (R)-enantiomer or (S)-enantiomer. Advantageously, the (R)-enantiomer may be chemically synthesised which may increase productivity. Advantageously, the naturally-occurring (S)-enantiomer may be obtained from a natural product, e.g. an organic product.

The composition for use according to the invention may be administered daily. Advantageously, daily administration of the composition provides for administration of the composition over a period of approximately 24 hours.

The composition for use according to the invention may be administered orally, topically and/or by inhalation. Advantageously, the different modes of administration enable systemic delivery when administered orally; local delivery when administered topically; and systemic and local delivery when administered by inhalation. The different modes of administration may also provide sustained or immediate release of the active ingredient.

The composition for use according to the invention may be a pharmaceutical composition or a food supplement.

The composition for use according to the invention may be administered orally, for example as a tablet, capsule, soft gel, liquid, syrup, suspension, powder, chew, lozenge, gum or bar. The composition for use according to the invention may be administered in foods or beverages. The composition for use according to the invention may be a dried or powdered composition for reconstitution before use with water or another suitable vehicle (e.g., milk, fruit juice).

The composition for use according to the invention may be administered topically, for example in the form of a solution, emulsion, ointment, cream, gel, plaster, patch or spray.

The composition for use according to the invention may be administered by inhalation, for example in the form of an aerosol.

The composition for use according to the invention may be administered as a daily dose comprising at least about 0.1 mg/kg, at least about 0.2 mg/kg, at least about 0.3 mg/kg, at least about 0.4 mg/kg, at least about 0.5 mg/kg, at least about 0.6 mg/kg, at least about 0.7 mg/kg, at least about 0.8 mg/kg, at least about 0.9 mg/kg, at least about 1 mg/kg of anatabine or a salt thereof.

The composition for use according to the invention may be administered as a daily dose comprising no more than about 10 mg/kg, no more than about 9 mg/kg, no more than about 8 mg/kg, no more than about 7 mg/kg, no more than about 6 mg/kg, no more than about 5 mg/kg, no more than about 4 mg/kg, no more than about 3 mg/kg, no more than about 2 mg/kg, no more than about 1.5 mg/kg of anatabine or a salt thereof.

The composition for use according to the invention may be administered as a daily dose comprising about 0.1 mg/kg to about 10 mg/kg, about 0.2 mg/kg to about 9 mg/kg, about 0.3 mg/kg to about 8 mg/kg, about 0.4 mg/kg to about 7 mg/kg, about 0.5 mg/kg to about 6 mg/kg, about 0.6 mg/kg to about 5 mg/kg, about 0.7 mg/kg to about 4 mg/kg, about 0.8 mg/kg to about 3 mg/kg, about 0.9 mg/kg to about 2 mg/kg, about 1 mg/kg to about 1.5 mg/kg of anatabine or a salt thereof. Preferably, the composition for use according to the invention may be administered as a daily dose comprising about 1 to about 2 mg/kg, preferably about 1.6 mg/kg of anatabine or a salt thereof. The daily dose may be administered at any range from the given endpoints.

The composition for use according to the invention may be administered as a daily dose comprising at least about 0.1 mg, at least about 0.5 mg, at least about 1 mg, at least about 2 mg, at least about 3 mg, at least about 4 mg, at least about 5 mg, at least about 6 mg, at least about 7 mg, at least about 8 mg, at least about 9 mg, at least about 10 mg, at least about 11 mg, at least about 12 mg, at least about 13 mg, at least about 14 mg, at least about 15 mg of anatabine or a salt thereof.

The composition for use according to the invention may be administered as a daily dose comprising no more than about 400 mg, no more than about 350 mg, no more than about 300 mg, no more than about 250 mg, no more than about 200 mg, no more than about 150 mg, no more than about 100 mg, no more than about 90 mg, no more than about 80 mg, no more than about 70 mg, no more than about 60 mg, no more than about 50 mg, no more than about 45 mg, no more than about 40 mg, no more than about 35 mg, no more than about 30 mg, no more than about 25 mg of anatabine or a salt thereof.

The composition for use according to the invention may be administered as a daily dose comprising about 0.1 mg to about 400 mg, about 0.5 mg to about 350 mg, 1 mg to about 300 mg, about 2 mg to about 250 mg, about 3 mg to about 200 mg, about 4 mg to about 150 mg, about 5 mg to about 100 mg, about 6 mg to about 90 mg, about 7 mg to about 80 mg, about 8 mg to about 70 mg, about 9 mg to about 60 mg, about 10 mg to about 50 mg, about 11 mg to about 45 mg, about 12 mg to about 40 mg, about 13 mg to about 35 mg, about 14 mg to about 30 mg, about 15 mg to about 25 mg of anatabine or a salt thereof.

The daily dose may comprise any range from the given endpoints. The daily doses of the present invention may enable effective doses of anatabine or a salt thereof without the risk of toxicity.

The daily dose may be administered as at least one fixed dose. The daily dose may be administered as one to twenty fixed doses.

The fixed dose may comprise at least about 0.1 mg, at least about 0.2 mg, at least about 0.3 mg, at least about 0.4 mg, at least about 0.5 mg, at least about 0.6 mg, at least about 0.7 mg, at least about 0.8 mg, at least about 0.9 mg, at least about 1 mg, at least about 2 mg, at least about 3 mg, at least about 4 mg, at least about 5 mg, at least about 6 mg, at least about 7 mg, at least about 8 mg, at least about 9 mg of anatabine or a salt thereof.

The fixed dose may comprise no more than about 100 mg, no more than about 95 mg, no more than about 90 mg, no more than about 85 mg, no more than about 80 mg, no more than about 75 mg, no more than about 70 mg, no more than about 65 mg, no more than about 60 mg, no more than about 55 mg, no more than about 50 mg, no more than about 45 mg, no more than about 40 mg, no more than about 35 mg, no more than about 30 mg, no more than about 25 mg, no more than about 20 mg, no more than about 15 mg of anatabine or a salt thereof.

The fixed dose may comprise about 0.1 mg to about 100 mg, about 0.2 mg to about 95 mg, about 0.3 mg to about 90 mg, about 0.4 mg to about 85 mg, about 0.5 mg to about 80 mg, about 0.6 mg to about 75 mg, about 0.7 mg to about 70 mg, about 0.8 mg to about 65 mg, about 0.9 mg to about 60 mg, about 1 mg to about 55 mg, about 2 mg to about 50 mg, about 3 mg to about 45 mg, about 4 mg to about 40 mg, about 5 mg to about 35 mg, about 6 mg to about 30 mg, about 7 mg to about 25 mg, about 8 mg to about 20 mg, about 9 mg to about 15 mg of anatabine or a salt thereof. The fixed dose may comprise any range from the given endpoints.

The effective concentration of anatabine or the salt thereof in a subject following administration may be at least about 1 μM, at least about 2 μM, at least about 3 μM, at least about 4 μM, at least about 5 μM, at least about 10 μM, at least about 20 μM, at least about 30 μM, at least about 40 μM, at least about 50 μM, at least about 100 μM, at least about 200 μM, at least about 300 μM, at least about 400 μM, at least about 500 μM, at least about 600 μM, at least about 700 μM, at least about 800 μM, at least about 900 μM, at least about 1000 μM, at least about 1100 μM, at least about 1200 μM, at least about 1300 μM, at least about 1400 μM, of anatabine or a salt thereof.

The effective concentration of anatabine or the salt thereof in the subject following administration may be no more than about 10000 μM, no more than about 9000 μM, no more than about 8000 μM, no more than about 7000 μM, no more than about 6000 μM, no more than about 5000 μM, no more than about 4500 μM, no more than about 4000 μM, no more than about 3500 μM, no more than about 3000 μM, no more than about 2900 μM, no more than about 2800 μM, no more than about 2700 μM, no more than about 2600 μM, no more than about 2500 μM, no more than about 2400 μM, no more than about 2300 μM, no more than about 2200 μM, no more than about 2100 μM, no more than about 2000 μM, no more than about 1900 μM, no more than about 1800 μM, no more than about 1700 μM, no more than about 1600 μM of anatabine or a salt thereof.

The effective concentration of anatabine or the salt thereof in the subject following administration may be about 1 μM to about 10000 μM, about 2 μM to about 9000 μM, about 3 μM to about 8000 μM, about 4 μM to about 7000 μM, about 5 μM to about 6000 μM, about 10 μM to about 5000 μM, about 20 μM to about 4500 μM, about 30 μM to about 4000 μM, about 40 μM to about 3500 μM, about 50 μM to about 3000 μM, about 100 μM to about 2900 μM, about 200 μM to about 2800 μM, about 300 μM to about 2700 μM, about 400 μM to about 2600 μM, about 500 μM to about 2500 μM, about 600 μM to about 2400 μM, about 700 μM to about 2300 μM, about 800 μM to about 2200 μM, about 900 μM to about 2100 μM, about 1000 μM to about 2000 μM, about 1100 μM to about 1900 μM, about 1200 μM to about 1800 μM, about 1300 μM to about 1700 μM, about 1400 μM to about 1600 μM of anatabine or a salt thereof.

The effective concentration of anatabine or the salt thereof in the subject following administration may comprise any range from the given endpoints. The compositions of the present invention may enable effective doses of anatabine or a salt thereof without the risk of toxicity.

According to a second aspect of the invention, there is provided a composition comprising anatabine or a salt thereof for use in the prophylactic treatment of a subject requiring NRF2 activation.

According to a third aspect of the invention, there is provided a use of anatabine or a salt thereof as a NRF2 activator.

According to further aspects of the invention, there is provided:

A method of activating NRF2 by administering anatabine or a salt thereof to a subject.

A method of increasing activity of NRF2 in a subject.

The use of anatabine or a salt thereof as an anti-inflammatory agent. The use of anatabine or a salt thereof as an anti-oxidant. The use of anatabine or a salt thereof as a food supplement for NRF2 activation.

Methods for promoting an anti-oxidative response; methods for improving an anti-inflammatory response; methods of improving cognitive performance; methods for building muscle. The methods may comprise steps of administering anatabine or a salt thereof to a subject.

A composition comprising anatabine or a salt thereof for use in the prophylactic activation of NRF2. Prophylactic activation of NRF2 comprises prophylactic activation of NRF2 and/or the NRF2 pathway.

A composition comprising anatabine or a salt thereof may be for use in the manufacture of a medicament. The medicament may be for use in the treatment of a NRF2-regulated disorder.

Anatabine or a salt thereof for use as a medicament.

A method of treatment comprising administration of a composition comprising anatabine or a salt thereof to a subject. The treatment may comprise the treatment of a NRF2-regulated disorder. The treatment may comprise steps of daily administration orally, topically, and/or by inhalation.

### DETAILED DESCRIPTION

Embodiments of the invention will now be described, by way of example only, with reference to the following figures:

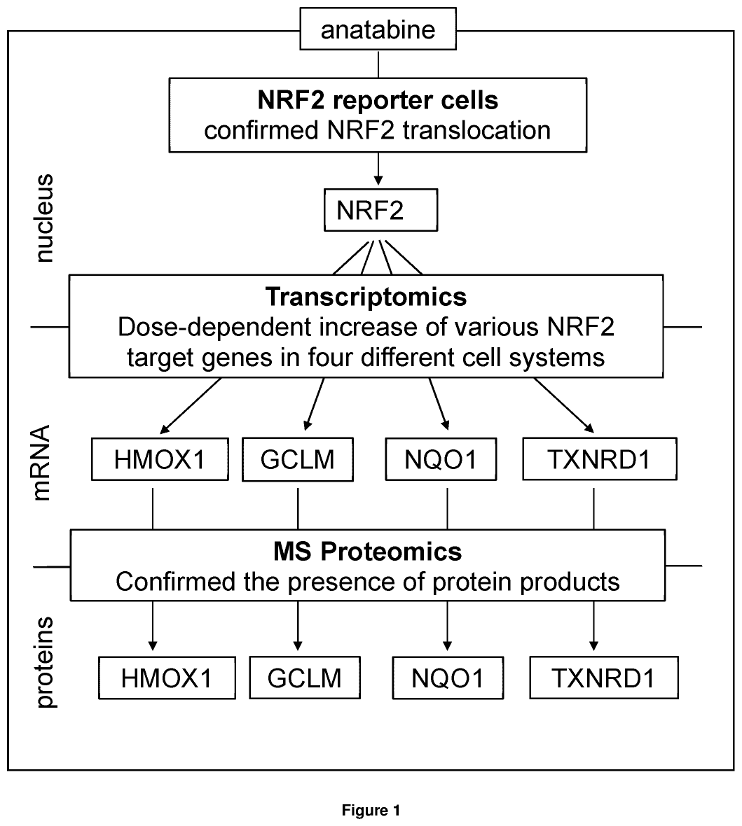

FIG. 1: a flow chart showing NRF2 regulation pathways;

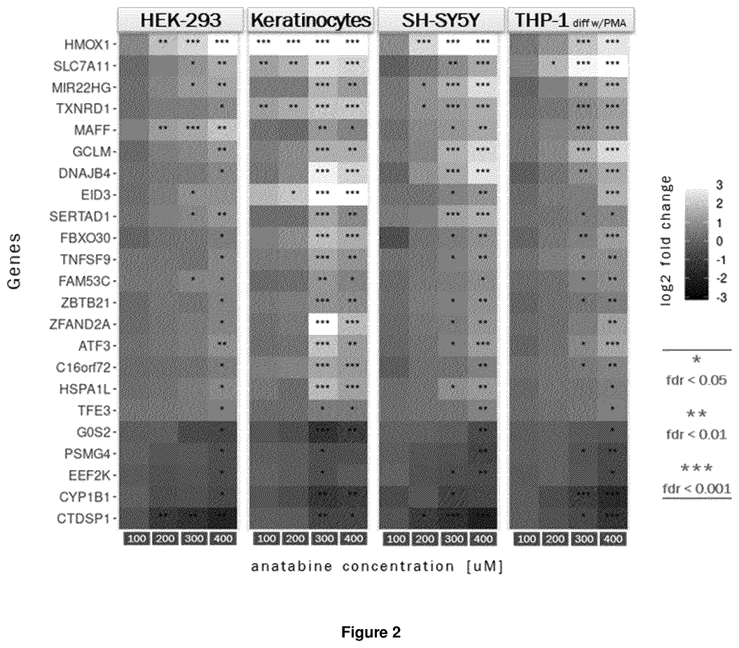

FIG. 2: a plot showing genes stimulated by anatabine arranged by descending median fold change taking into consideration all anatabine treatment concentrations;

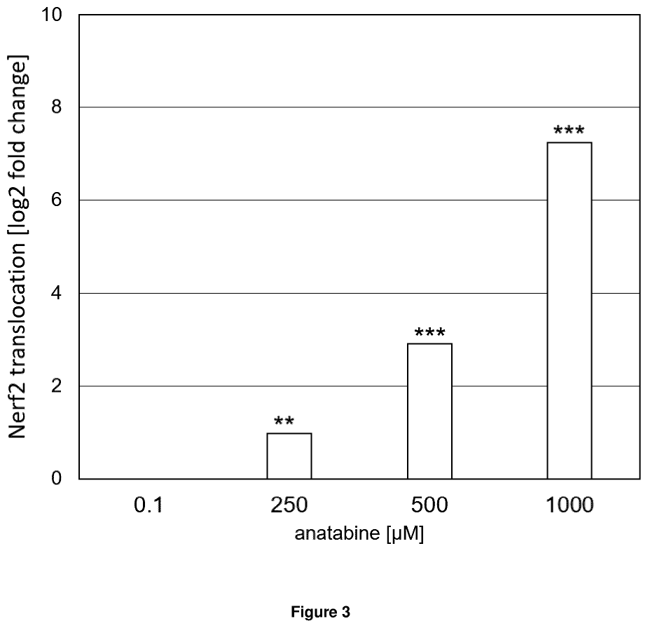

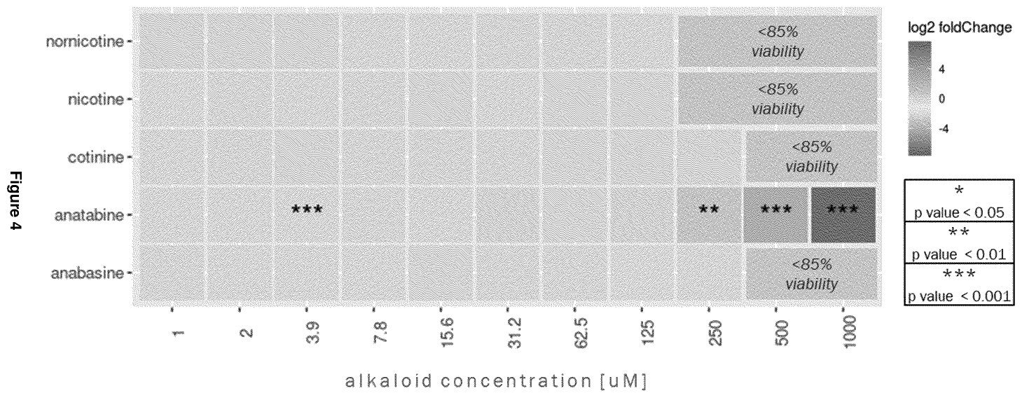

FIG. 3: a bar graph showing NRF2 translocation for anatabine;

FIG. 4: a panel graph showing NRF2 translocation and cell viability for a selection of alkaloids including anatabine, nornicotine, nicotine, cotinine and anabasine;

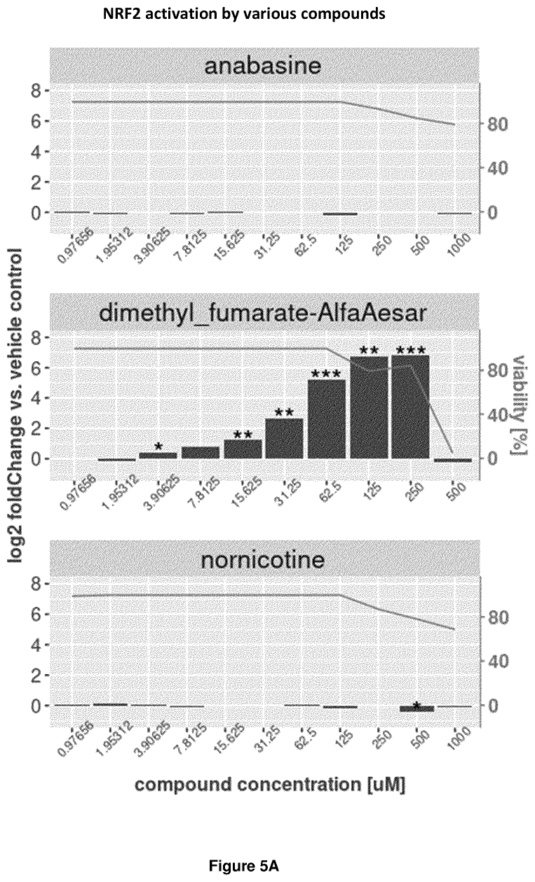

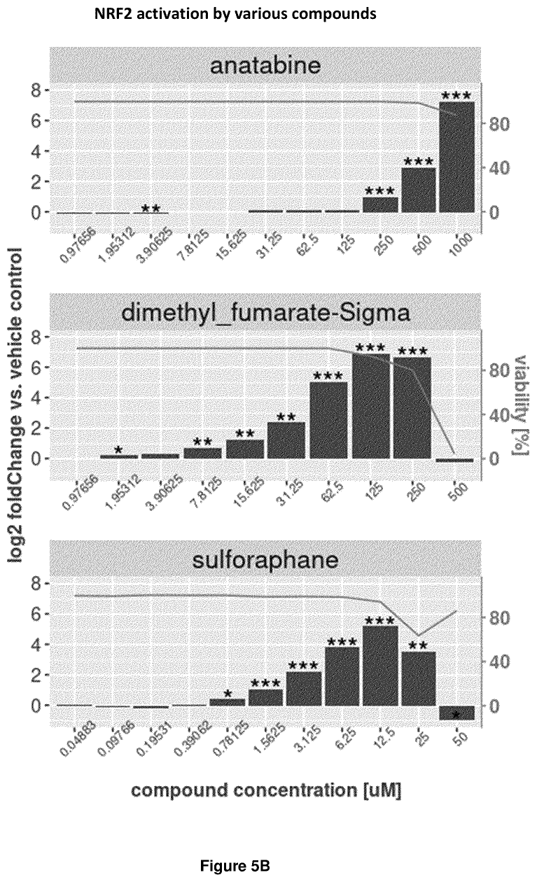

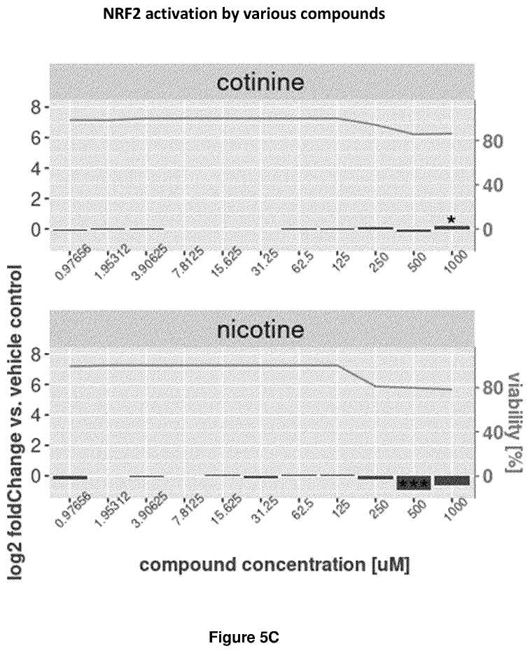

FIG. 5: a bar and line graph showing NRF2 translocation presented as log2 fold change compared to vehicle control (bars) and cell viability measured with ATP assay and presented as a percentage of the vehicle control (line) for anatabine, nornicotine, nicotine, cotinine, anabasine, dimethyl fumarate (×2) and sulforaphane.

### MATERIALS AND METHODS

### Cell Culture and Compound Testing

To investigate the signalling pathways triggered by anatabine, 4 cell systems (HEK-293, SH-SY5Y, primary human keratinocytes, THP-1 differentiated with PMA) were treated with 4 concentrations of anatabine (100, 200, 300, 400 μM) for 6 hours, after which transcriptomics measurements were performed.

HEK-293 is a human embryonic kidney cell line devoid of nicotinic receptors. It can be relatively easily transfected and therefore used as a tool to express various subtypes of nicotinic receptors or reporter genes such as NRF2-driven luciferase, which were also used in this study. HEK-293 were seeded in a cell culture medium consisting of EMEM, 10% FBS, 1% NEAA, 1% Sodium Pyruvate and 1% PenStrep. 60,000 cells were seeded per well of a 96-well plate.

SH-SY5Y is a human bone marrow neuroblastoma cell line that has been used by researchers to investigate the potential anti-inflammatory effect of anatabine. SH-SY5Y were seeded in a cell culture medium consisting of 50% EMEM and 50% Ham's F12 nutrient and including 10% FBS, 1% PenStrep and 1% L-glutamine. 15,000 cells were seeded per well of a 96-well plate.

Primary human keratinocytes have been included as a relevant cell system for skin. The batch of keratinocytes was selected in order to have healthy, non-smoker, 20-60 years old, Caucasian donors. The keratinocytes were seeded in a cell culture medium consisting of KGM-Gold Keratinocyte Growth Medium. 17,500 cells were seeded per well of a 96-well plate.

The human leukaemia-derived monocytic cell line THP-1, is the most widely used in vitro model for primary human macrophage investigations. THP-1 cells were seeded in a cell culture medium consisting of RPMI, 10% FBS and 1% PenStrep and in the presence of 40 ng/ml phorbol 12-myristate 13-acetate (PMA). 100,000 cells were seeded per well of a 96-well plate, having a total volume of 200 μl per well. 48 hours later the medium was changed to the same culture medium but without PMA, and the culture was left to rest for 24 h before treatment.

All four cell systems were seeded in 96 well plates at a total volume of 190 μl per well to allow for a 20× compound stimulation with a volume of 10 μl. 24 hours after seeding—or in the case of THP-1, 24 h after changing to non-PMA medium—four cell systems were stimulated with anatabine (prepared at 20× the corresponding target concentration) to obtain a final molar concentration of 100, 200, 300, 400 μM anatabine.

In all experiments the racemic anatabine free base was used which has a MW of 160.22 g per mole. The compound came from a single production batch, hereinafter referred to as ‘anatabine’.

### Cell Viability

Anatabine concentrations were selected so that no less than an 80% viability was observed after 24 hours of exposure, which for anatabine free base was translated into no more than 400 μM target concentration. Viability has been assessed with CellTiterGlo, measuring ATP content and comparing each sample that had treatment with its relevant control (cells treated with cell culture medium only).

### Transcriptomics Data Generation

In order to generate the transcriptomics data, the four cell systems were exposed to various concentrations of anatabine (100, 200, 300, 400 μM) for 6 hours. The transcriptome of the cells was analysed by a microarray-based analysis.

The cells were lysed using RLT buffer (Qiagen Hilden, Germany) for all cell types except the keratinocytes which were lysed with Qiazol lysis reagent (Qiagen). RNA isolation was performed with Rneasy Micro Kit (Qiagen). RNA quantification was assessed with a Nanodrop 1000 (ThermoFisher Waltham, MA, USA) and quality of the total RNA was verified acquiring a profile with a 2100 Bioanalyzer (Agilent Technologies, Santa Clara, CA, USA), requiring an RIN number greater than 6.0. 50 ng of total RNA were processed as described in the Tecan/Nugen Ovation RNA Amplification system V2 (Tecan Männedorf, Switzerland). Human Genome U133 Plus 2.0 array were used for hybridization on a ThermoFisher Oven 645, washed on ThermoFisher GeneChip™ Fluidics Stations 450Dx (protocol FS450-0004) and scanned on a ThermoFisher GeneChip™ Scanner 3000 7G.

Quality control check of all chips was done with the Bioconductor affyPLM R package version 1.64. Following quality control procedures, differential gene expression analysis was performed using the Bioconductor Limma R package version 3.44.3. Pairwise comparisons at the gene level, called systems response profiles (SRP), were computed by comparing each concentration-treatment with its respective vehicle control. Genes with a false discovery rate (FDR) below 0.05 were considered differentially expressed genes (DEG). SRPs including all genes (˜18,000) were further leveraged in downstream p-value threshold-free gene set enrichment analysis as described below in section “Gene Set Enrichment Analysis”.

### Gene Set Enrichment Analysis

To investigate the biological significance of the transcriptomic results, Gene Set Enrichment Analysis (GSEA) was employed, a computational method to determine whether an a priori defined set of genes shows statistically significant concordant differences between two biological states (Subramanian, A., et al., Gene set enrichment analysis: a knowledge-based approach for interpreting genome-wide expression profiles. Proc Natl Acad Sci USA, 2005. 102(43): p. 15545-50). As a priori knowledge gene sets, two gene sets collections named “C2-CP” and “C3-TFT” were retrieved from the Molecular Signatures Database (MsigDB), version 7.1 (Liberzon et al., 2015). C2-CP” is the Canonical Pathways (CP) collection of the C2 MsigDB category, which contains all the curated gene sets. “C3-TFT” is the Transcription Factor Targets (TFT) collection of the C3 MsigDB category, which contains all the regulatory target gene sets.

Gene set enrichment analyses revealed a significant enrichment of genes representative of the Nuclear factor (erythroid-derived 2)-like 2 (NFE2L2 or NRF2)-signalling pathway.

### Data Independent Acquisition (DIA) Mass Spectrometry Proteomics Data Generation

To confirm the increased expression of target genes of NRF2 transcription factor at the protein level, proteins were profiled by untargeted mass spectrometry in PMA-differentiated THP-1 cell lysates after anatabine treatment (100, 200, 300, 400 μM) for 24 hours. It was found that intracellular protein products of NRF2 target genes, specifically HMOX1, GCLM, NQO1 and TXNRD1, were all significantly expressed (adjusted p-value<0.05).

Samples were processed using PreOmics iST kit (PreOmics GmbH, Planegg/Martinsried, Germany) as described in the manufacturer's protocol. Briefly, 100 μl of PreOmics lyse buffer were added to the cells, samples were incubated at 90° C. for 10 min and sonified for 30 sec with a sonifier (Brandson, Danbury, Connecticut, USA) at 10% amplitude. Protein amount was determined using the Pierce 660 nm protein assay (Pierce Biotechnology, Rockford, USA) following the manufacturer's protocol. Samples were normalized to 0.5 μg/μl and 40 μg was further processed with PreOmics iST kit with a 3 h trypsin digestion. Peptides were purified on the cartridge, dried overnight on a vacuum concentrator (Martin Christ, Germany) and re-suspended in 50 μL of LC Solution (Biognosys AG, Switzerland). 1 μL of iRT reference peptides (Biognosys AG, Switzerland) was added to 19 μL of processed sample. Samples were analysed with an Easy 1000 nanoLC (ThermoFisher Scientific) connected online to a Q-Exactive (ThermoFisher Scientific). 2 μL of peptide mixture were separated on a 0.75×500 mm, 1.7 μm C18 column (ThermoFisher Scientific) using solvent A: 1% acetonitrile/99% water/0.1 formic acid and a 115 min gradient from 5% to 35% of solvent B: 95% acetonitrile/5% water/0.1 formic acid at 200 nL/min.

Data were acquired on a Q-Exactive in data-independent acquisition (DIA) mode: MS1 scan at a 140 k resolution was followed by 23 custom MS/MS m/z windows at a 35 k resolution. Data were processed with Spectronaut Pulsar (v. 13.8.190930.43655 Biognosys AG, Switzerland) using the DirectDIA feature.

SRPs were computed by comparing each concentration-treatment with its respective vehicle control, in a similar fashion with transcriptomics data generation, as described herein under “Transcriptomics data generation”.

### NRF2 Luciferase Assay

The activation of NRF2 by anatabine was further confirmed using an NRF2 luciferase reporter cell line assay, which showed a concentration-dependent rise of fluorescence after treatment with anatabine.

A HEK-293 NRF2/ARE luciferase reporter cell line was acquired from Signosis (USA). According to Signosis, this NRF2 luciferase reporter stable cell line had been stably transfected with pTA-ARE-luciferase reporter vector, which contains 4 repeats of antioxidant response binding sites, a minimal promoter upstream of the firefly luciferase coding region, along with a hygromycin expression vector. Following selection, the hygromycin resistant clones were subsequently screened for TBHQ-induced luciferase activity. The clone with the highest fold induction was selected and expanded to produce this stable cell line.

4, 8 or 24 hours after stimulation of the NRF2 reporter cells luminescence was measured using a FLUOstar Omega plate reader.

### Results

The results show that anatabine treatment activates the transcription factor NRF2 pathways summarised in FIG. 1.

FIG. 2 and Table 1 below show the stimulation of genes by anatabine in all of the cell systems.

TABLE 1
List of genes and their respective stimulation by anatabine

| | Cell | Anatabine | Fold | FDR adjusted | p-value |
|---|---|---|---|---|---|
| Gene | system | [μM] | Change | p-value | symbol |
| ATF3 | HEK293 | 100 | 0.244335938 | 0.815909325 | |
| ATF3 | HEK293 | 200 | 0.375074402 | 0.390202739 | |
| ATF3 | HEK293 | 300 | 0.517611093 | 0.138500939 | |
| ATF3 | HEK293 | 400 | 0.928572024 | 0.009641592 | ** |
| ATF3 | KEAT | 100 | 0.210600237 | 0.729191055 | |
| ATF3 | KEAT | 200 | 0.316889327 | 0.463286153 | |
| ATF3 | KEAT | 300 | 1.893235458 | 0.000682851 | *** |
| ATF3 | KEAT | 400 | 1.184751159 | 0.009735593 | ** |
| ATF3 | SHSY5Y | 100 | 0.047014281 | 0.987067063 | |
| ATF3 | SHSY5Y | 200 | 0.303777243 | 0.762696277 | |
| ATF3 | SHSY5Y | 300 | 0.620681334 | 0.024955738 | * |
| ATF3 | SHSY5Y | 400 | 1.206492656 | 0.000816334 | *** |
| ATF3 | THP1 | 100 | −0.026476575 | 0.982143464 | |
| ATF3 | THP1 | 200 | 0.250737196 | 0.77604963 | |
| ATF3 | THP1 | 300 | 0.784888692 | 0.017131817 | * |
| ATF3 | THP1 | 400 | 1.355409557 | 0.000591373 | *** |
| C16orf72 | HEK293 | 100 | 0.155388586 | 0.819357227 | |
| C16orf72 | HEK293 | 200 | 0.207722698 | 0.545418107 | |
| C16orf72 | HEK293 | 300 | 0.266634639 | 0.271513525 | |
| C16orf72 | HEK293 | 400 | 0.531790927 | 0.047615584 | * |
| C16orf72 | KEAT | 100 | 0.569297004 | 0.280831541 | |
| C16orf72 | KEAT | 200 | 0.689404087 | 0.052271922 | |
| C16orf72 | KEAT | 300 | 1.380080533 | 0.000140636 | *** |
| C16orf72 | KEAT | 400 | 1.1360643 | 0.000862424 | *** |
| C16orf72 | SHSY5Y | 100 | −0.199112817 | 0.975446513 | |
| C16orf72 | SHSY5Y | 200 | 0.251405764 | 0.824008751 | |
| C16orf72 | SHSY5Y | 300 | 0.283493652 | 0.192760643 | |
| C16orf72 | SHSY5Y | 400 | 0.666275851 | 0.009331899 | ** |
| C16orf72 | THP1 | 100 | 0.185078962 | 0.902448724 | |
| C16orf72 | THP1 | 200 | 0.330575329 | 0.544690546 | |
| C16orf72 | THP1 | 300 | 0.474164205 | 0.037702383 | * |
| C16orf72 | THP1 | 400 | 0.693572695 | 0.003224342 | ** |
| CTDSP1 | HEK293 | 100 | −0.413939636 | 0.782459077 | |
| CTDSP1 | HEK293 | 200 | −1.148361889 | 0.007762025 | ** |
| CTDSP1 | HEK293 | 300 | −1.375356943 | 0.002614092 | ** |
| CTDSP1 | HEK293 | 400 | −1.809922191 | 0.003945554 | ** |
| CTDSP1 | KEAT | 100 | −0.1511869 | 0.811788092 | |
| CTDSP1 | KEAT | 200 | −0.1977111 | 0.678873 | |
| CTDSP1 | KEAT | 300 | −0.805422766 | 0.0046651 | ** |
| CTDSP1 | KEAT | 400 | −0.769103166 | 0.045864996 | * |
| CTDSP1 | SHSY5Y | 100 | −0.435309849 | 0.975446513 | |
| CTDSP1 | SHSY5Y | 200 | −1.065215772 | 0.034846485 | * |
| CTDSP1 | SHSY5Y | 300 | −1.405452244 | 0.000383717 | *** |
| CTDSP1 | SHSY5Y | 400 | −1.962763922 | 0.000340109 | *** |
| CTDSP1 | THP1 | 100 | −0.174355134 | 0.902677217 | |
| CTDSP1 | THP1 | 200 | −0.425246454 | 0.543196044 | |
| CTDSP1 | THP1 | 300 | −0.714136447 | 0.012287617 | * |
| CTDSP1 | THP1 | 400 | −1.29577929 | 0.000321361 | *** |
| CYP1B1 | HEK293 | 100 | −0.124608761 | 0.856612339 | |
| CYP1B1 | HEK293 | 200 | −0.55922128 | 0.236775351 | |
| CYP1B1 | HEK293 | 300 | −0.453327403 | 0.24882832 | |
| CYP1B1 | HEK293 | 400 | −0.705402561 | 0.026528237 | * |
| CYP1B1 | KEAT | 100 | −0.367957173 | 0.558488229 | |
| CYP1B1 | KEAT | 200 | −0.609688918 | 0.221087128 | |
| CYP1B1 | KEAT | 300 | −1.240374409 | 0.001140419 | ** |
| CYP1B1 | KEAT | 400 | −1.021334694 | 0.006437621 | ** |
| CYP1B1 | SHSY5Y | 100 | −0.65182924 | 0.975446513 | |
| CYP1B1 | SHSY5Y | 200 | −0.324004281 | 0.868413551 | |
| CYP1B1 | SHSY5Y | 300 | −0.848563203 | 0.045580313 | * |
| CYP1B1 | SHSY5Y | 400 | −0.727248782 | 0.097095343 | |
| CYP1B1 | THP1 | 100 | −0.178682308 | 0.898588673 | |
| CYP1B1 | THP1 | 200 | −0.607012141 | 0.219856801 | |
| CYP1B1 | THP1 | 300 | −0.973602471 | 0.000358803 | *** |
| CYP1B1 | THP1 | 400 | −1.573073456 | 9.64127E−05 | *** |
| DNAJB4 | HEK293 | 100 | 0.241629243 | 0.782459077 | |
| DNAJB4 | HEK293 | 200 | 0.419951286 | 0.361876782 | |
| DNAJB4 | HEK293 | 300 | 0.46793026 | 0.126547401 | |
| DNAJB4 | HEK293 | 400 | 0.791666842 | 0.017792306 | * |
| DNAJB4 | KEAT | 100 | 0.406888189 | 0.507989742 | |
| DNAJB4 | KEAT | 200 | 0.824824522 | 0.052271922 | |
| DNAJB4 | KEAT | 300 | 2.757963821 | 1.85786E−05 | *** |
| DNAJB4 | KEAT | 400 | 2.194779017 | 7.32868E−05 | *** |
| DNAJB4 | SHSY5Y | 100 | 0.001154954 | 0.998883795 | |
| DNAJB4 | SHSY5Y | 200 | 0.971292486 | 0.081227602 | |
| DNAJB4 | SHSY5Y | 300 | 1.465659294 | 0.00062465 | *** |
| DNAJB4 | SHSY5Y | 400 | 2.495625645 | 2.44483E−05 | *** |
| DNAJB4 | THP1 | 100 | −0.173503505 | 0.902448724 | |
| DNAJB4 | THP1 | 200 | 0.333398927 | 0.617881626 | |
| DNAJB4 | THP1 | 300 | 0.917903283 | 0.002670205 | ** |
| DNAJB4 | THP1 | 400 | 1.516861288 | 0.000104299 | *** |
| EEF2K | HEK293 | 100 | −0.249956822 | 0.790844151 | |
| EEF2K | HEK293 | 200 | −0.493276627 | 0.236775351 | |
| EEF2K | HEK293 | 300 | −0.381930697 | 0.256490906 | |
| EEF2K | HEK293 | 400 | −0.594094298 | 0.039734693 | * |
| EEF2K | KEAT | 100 | −0.070338973 | 0.919026102 | |
| EEF2K | KEAT | 200 | −0.216376502 | 0.652010449 | |
| EEF2K | KEAT | 300 | −0.562453399 | 0.039600955 | * |
| EEF2K | KEAT | 400 | −0.272901931 | 0.408263966 | |
| EEF2K | SHSY5Y | 100 | −0.073085944 | 0.980731975 | |
| EEF2K | SHSY5Y | 200 | −0.461748604 | 0.423781847 | |
| EEF2K | SHSY5Y | 300 | −0.571150534 | 0.024998163 | * |
| EEF2K | SHSY5Y | 400 | −0.660840023 | 0.007258814 | ** |
| EEF2K | THP1 | 100 | −0.226382367 | 0.898588673 | |
| EEF2K | THP1 | 200 | −0.064056856 | 0.918964087 | |
| EEF2K | THP1 | 300 | −0.34194919 | 0.069524533 | |
| EEF2K | THP1 | 400 | −0.72453575 | 0.010638273 | * |
| EID3 | HEK293 | 100 | 0.183314329 | 0.889975293 | |
| EID3 | HEK293 | 200 | 0.53449219 | 0.399923406 | |
| EID3 | HEK293 | 300 | 0.924745315 | 0.046542515 | * |
| EID3 | HEK293 | 400 | 0.952751662 | 0.099452481 | |
| EID3 | KEAT | 100 | 1.694363819 | 0.098060861 | |
| EID3 | KEAT | 200 | 1.995199748 | 0.012552261 | * |
| EID3 | KEAT | 300 | 3.640613984 | 4.36671E−05 | *** |
| EID3 | KEAT | 400 | 3.120255431 | 0.000254389 | *** |
| EID3 | SHSY5Y | 100 | 0.29133209 | 0.975446513 | |
| EID3 | SHSY5Y | 200 | 0.31557321 | 0.729595981 | |
| EID3 | SHSY5Y | 300 | 0.686833408 | 0.022916491 | * |
| EID3 | SHSY5Y | 400 | 1.050134131 | 0.002584393 | ** |
| EID3 | THP1 | 100 | 0.11206887 | 0.914436878 | |
| EID3 | THP1 | 200 | 0.467926468 | 0.543196044 | |
| EID3 | THP1 | 300 | 0.569806645 | 0.054105403 | |
| EID3 | THP1 | 400 | 1.614551975 | 0.000136362 | *** |
| FAM53C | HEK293 | 100 | 0.379212648 | 0.782459077 | |
| FAM53C | HEK293 | 200 | 0.422988347 | 0.361332002 | |
| FAM53C | HEK293 | 300 | 0.696937104 | 0.041107349 | * |
| FAM53C | HEK293 | 400 | 0.736672925 | 0.028591177 | * |
| FAM53C | KEAT | 100 | 0.1268935 | 0.815841885 | |
| FAM53C | KEAT | 200 | 0.219446629 | 0.632525092 | |
| FAM53C | KEAT | 300 | 1.0672081 | 0.001218428 | ** |
| FAM53C | KEAT | 400 | 0.685271084 | 0.010795639 | * |
| FAM53C | SHSY5Y | 100 | 0.121190019 | 0.975446513 | |
| FAM53C | SHSY5Y | 200 | 0.428260598 | 0.553104877 | |
| FAM53C | SHSY5Y | 300 | 0.542712432 | 0.050498086 | |
| FAM53C | SHSY5Y | 400 | 0.873045748 | 0.010743084 | * |
| FAM53C | THP1 | 100 | 0.144002479 | 0.902448724 | |
| FAM53C | THP1 | 200 | 0.251514465 | 0.63331721 | |
| FAM53C | THP1 | 300 | 0.629623452 | 0.011530688 | * |
| FAM53C | THP1 | 400 | 0.87293366 | 0.002008271 | * * |
| FBXO30 | HEK293 | 100 | 0.001538537 | 0.999003559 | |
| FBXO30 | HEK293 | 200 | 0.287215407 | 0.462780698 | |
| FBXO30 | HEK293 | 300 | 0.358612614 | 0.186453509 | |
| FBXO30 | HEK293 | 400 | 0.594888524 | 0.031958425 | * |
| FBXO30 | KEAT | 100 | 0.61899484 | 0.280831541 | |
| FBXO30 | KEAT | 200 | 0.880702556 | 0.052271922 | |
| FBXO30 | KEAT | 300 | 1.831761545 | 3.41818E−05 | *** |
| FBXO30 | KEAT | 400 | 1.526189548 | 0.000199737 | *** |
| FBXO30 | SHSY5Y | 100 | −0.519530841 | 0.975446513 | |
| FBXO30 | SHSY5Y | 200 | 0.31530171 | 0.58736155 | |
| FBXO30 | SHSY5Y | 300 | 0.547452223 | 0.016725533 | * |
| FBXO30 | SHSY5Y | 400 | 0.926854377 | 0.00187309 | ** |
| FBXO30 | THP1 | 100 | 0.002855954 | 0.998930763 | |
| FBXO30 | THP1 | 200 | 0.426980029 | 0.335216476 | |
| FBXO30 | THP1 | 300 | 0.659491161 | 0.003054116 | ** |
| FBXO30 | THP1 | 400 | 1.224348283 | 0.000158947 | *** |
| G0S2 | HEK293 | 100 | −0.159025291 | 0.855231034 | |
| G0S2 | HEK293 | 200 | −0.111422213 | 0.898114441 | |
| G0S2 | HEK293 | 300 | −0.68535077 | 0.105680283 | |
| G0S2 | HEK293 | 400 | −0.729168536 | 0.034432922 | * |
| G0S2 | KEAT | 100 | −0.351613895 | 0.557219348 | |
| G0S2 | KEAT | 200 | −0.550839945 | 0.139878748 | |
| G0S2 | KEAT | 300 | −1.36103243 | 0.000134492 | *** |
| G0S2 | KEAT | 400 | −1.015740843 | 0.001414797 | ** |
| G0S2 | SHSY5Y | 100 | −0.064796709 | 0.982597848 | |
| G0S2 | SHSY5Y | 200 | −0.074078406 | 0.955926761 | |
| G0S2 | SHSY5Y | 300 | −0.162088678 | 0.604104966 | |
| G0S2 | SHSY5Y | 400 | −0.66232488 | 0.00886052 | ** |
| G0S2 | THP1 | 100 | −0.161340071 | 0.902448724 | |
| G0S2 | THP1 | 200 | −0.074725767 | 0.912519196 | |
| G0S2 | THP1 | 300 | −0.267397502 | 0.15999346 | |
| G0S2 | THP1 | 400 | −0.367511855 | 0.046204834 | * |
| GCLM | HEK293 | 100 | 0.108601168 | 0.865003549 | |
| GCLM | HEK293 | 200 | 0.597551193 | 0.199661479 | |
| GCLM | HEK293 | 300 | 0.765186654 | 0.050419523 | |
| GCLM | HEK293 | 400 | 1.165417002 | 0.007554107 | ** |
| GCLM | KEAT | 100 | 0.569812028 | 0.3687932 | |
| GCLM | KEAT | 200 | 0.854920027 | 0.061588203 | |
| GCLM | KEAT | 300 | 1.581599473 | 0.000179852 | *** |
| GCLM | KEAT | 400 | 1.566674683 | 0.001065638 | ** |
| GCLM | SHSY5Y | 100 | 0.390673808 | 0.975446513 | |
| GCLM | SHSY5Y | 200 | 0.705310191 | 0.156266428 | |
| GCLM | SHSY5Y | 300 | 1.62858117 | 0.00062465 | *** |
| GCLM | SHSY5Y | 400 | 2.291523194 | 1.17566E−05 | *** |
| GCLM | THP1 | 100 | 0.299154274 | 0.898588673 | |
| GCLM | THP1 | 200 | 0.781561764 | 0.308359955 | |
| GCLM | THP1 | 300 | 1.64497739 | 0.000327745 | *** |
| GCLM | THP1 | 400 | 2.359855449 | 3.71465E−05 | *** |
| HMOX1 | HEK293 | 100 | 0.774535728 | 0.782459077 | |
| HMOX1 | HEK293 | 200 | 1.877471888 | 0.007762025 | ** |
| HMOX1 | HEK293 | 300 | 2.269993466 | 0.000336978 | *** |
| HMOX1 | HEK293 | 400 | 3.142462413 | 0.000134805 | *** |
| HMOX1 | KEAT | 100 | 3.913929446 | 0.000202381 | *** |
| HMOX1 | KEAT | 200 | 4.283025633 | 2.08257E−05 | *** |
| HMOX1 | KEAT | 300 | 5.732672815 | 4.28088E−06 | *** |
| HMOX1 | KEAT | 400 | 5.632940574 | 1.83795E−06 | *** |
| HMOX1 | SHSY5Y | 100 | 0.869897384 | 0.975446513 | |
| HMOX1 | SHSY5Y | 200 | 2.286263828 | 0.000379437 | *** |
| HMOX1 | SHSY5Y | 300 | 3.747636658 | 9.2405E−08 | *** |
| HMOX1 | SHSY5Y | 400 | 5.505529832 | 1.7779E−07 | *** |
| HMOX1 | THP1 | 100 | 0.437803608 | 0.865461777 | |
| HMOX1 | THP1 | 200 | 0.988259181 | 0.05366415 | |
| HMOX1 | THP1 | 300 | 1.964370077 | 5.16219E−05 | *** |
| HMOX1 | THP1 | 400 | 2.547943481 | 1.52231E−06 | *** |
| HSPA1L | HEK293 | 100 | 0.187882765 | 0.824727651 | |
| HSPA1L | HEK293 | 200 | 0.296069917 | 0.574561941 | |
| HSPA1L | HEK293 | 300 | 0.529274742 | 0.138245405 | |
| HSPA1L | HEK293 | 400 | 0.784082254 | 0.021506893 | * |
| HSPA1L | KEAT | 100 | 0.413637155 | 0.66819208 | |
| HSPA1L | KEAT | 200 | 0.145406367 | 0.785430035 | |
| HSPA1L | KEAT | 300 | 1.768548895 | 0.000134492 | *** |
| HSPA1L | KEAT | 400 | 1.313894684 | 0.00071501 | *** |
| HSPA1L | SHSY5Y | 100 | 0.175775969 | 0.975446513 | |
| HSPA1L | SHSY5Y | 200 | 0.274836291 | 0.766039791 | |
| HSPA1L | SHSY5Y | 300 | 0.915701493 | 0.013532764 | * |
| HSPA1L | SHSY5Y | 400 | 1.134881723 | 0.003149275 | ** |
| HSPA1L | THP1 | 100 | 0.053745654 | 0.976419068 | |
| HSPA1L | THP1 | 200 | 0.323631936 | 0.748845313 | |
| HSPA1L | THP1 | 300 | 0.334133245 | 0.314447265 | |
| HSPA1L | THP1 | 400 | 0.563928708 | 0.04350904 | * |
| MAFF | HEK293 | 100 | 0.620293757 | 0.782459077 | |
| MAFF | HEK293 | 200 | 1.217146908 | 0.008590473 | ** |
| MAFF | HEK293 | 300 | 1.422400754 | 0.00075169 | *** |
| MAFF | HEK293 | 400 | 1.928667262 | 0.001827785 | ** |
| MAFF | KEAT | 100 | 0.288626407 | 0.754141202 | |
| MAFF | KEAT | 200 | 0.136882995 | 0.808966277 | |
| MAFF | KEAT | 300 | 1.12750079 | 0.001888725 | ** |
| MAFF | KEAT | 400 | 0.708550342 | 0.015342019 | * |
| MAFF | SHSY5Y | 100 | 0.219314351 | 0.975446513 | |
| MAFF | SHSY5Y | 200 | 0.786039152 | 0.394276591 | |
| MAFF | SHSY5Y | 300 | 1.06277312 | 0.016725533 | * |
| MAFF | SHSY5Y | 400 | 1.451362301 | 0.002560367 | ** |
| MAFF | THP1 | 100 | 0.308286275 | 0.898588673 | |
| MAFF | THP1 | 200 | 0.661236965 | 0.216435893 | |
| MAFF | THP1 | 300 | 0.932660996 | 0.000653389 | *** |
| MAFF | THP1 | 400 | 1.334654537 | 0.000106874 | *** |
| MIR22HG | HEK293 | 100 | 0.242209608 | 0.79645812 | |
| MIR22HG | HEK293 | 200 | 0.720816137 | 0.376706404 | |
| MIR22HG | HEK293 | 300 | 1.13468897 | 0.029643893 | * |
| MIR22HG | HEK293 | 400 | 1.371412273 | 0.004230311 | ** |
| MIR22HG | KEAT | 100 | 0.496482475 | 0.480944988 | |
| MIR22HG | KEAT | 200 | 0.510785794 | 0.29086815 | |
| MIR22HG | KEAT | 300 | 1.634023536 | 0.000372826 | *** |
| MIR22HG | KEAT | 400 | 1.170954054 | 0.002683431 | ** |
| MIR22HG | SHSY5Y | 100 | 0.409349542 | 0.975446513 | |
| MIR22HG | SHSY5Y | 200 | 1.012532856 | 0.034846485 | * |
| MIR22HG | SHSY5Y | 300 | 1.589199276 | 0.00062465 | *** |
| MIR22HG | SHSY5Y | 400 | 2.378956543 | 6.55193E−05 | *** |
| MIR22HG | THP1 | 100 | 0.01066619 | 0.996791053 | |
| MIR22HG | THP1 | 200 | 0.638123497 | 0.543196044 | |
| MIR22HG | THP1 | 300 | 1.183701092 | 0.006700636 | ** |
| MIR22HG | THP1 | 400 | 1.725136138 | 0.000272688 | *** |
| PSMG4 | HEK293 | 100 | −0.096825005 | 0.893817477 | |
| PSMG4 | HEK293 | 200 | −0.328981961 | 0.463789941 | |
| PSMG4 | HEK293 | 300 | −0.284541304 | 0.279748469 | |
| PSMG4 | HEK293 | 400 | −0.555199184 | 0.037253422 | * |
| PSMG4 | KEAT | 100 | −0.156727942 | 0.830473134 | |
| PSMG4 | KEAT | 200 | −0.216025201 | 0.657993817 | |
| PSMG4 | KEAT | 300 | −0.545072518 | 0.026927265 | * |
| PSMG4 | KEAT | 400 | −0.529890735 | 0.058103753 | |
| PSMG4 | SHSY5Y | 100 | −0.169139621 | 0.975446513 | |
| PSMG4 | SHSY5Y | 200 | −0.377672871 | 0.53001635 | |
| PSMG4 | SHSY5Y | 300 | −0.457174327 | 0.053513046 | |
| PSMG4 | SHSY5Y | 400 | −0.802828796 | 0.002654955 | ** |
| PSMG4 | THP1 | 100 | −0.150773668 | 0.902448724 | |
| PSMG4 | THP1 | 200 | −0.058334411 | 0.940105278 | |
| PSMG4 | THP1 | 300 | −0.411434314 | 0.041680792 | * |
| PSMG4 | THP1 | 400 | −0.713536052 | 0.002660843 | ** |
| SERTAD1 | HEK293 | 100 | 0.302636234 | 0.782459077 | |
| SERTAD1 | HEK293 | 200 | 0.620779618 | 0.241293325 | |
| SERTAD1 | HEK293 | 300 | 0.768122032 | 0.046542515 | * |
| SERTAD1 | HEK293 | 400 | 1.05378144 | 0.009641592 | ** |
| SERTAD1 | KEAT | 100 | 0.194686214 | 0.779945411 | |
| SERTAD1 | KEAT | 200 | 0.214703791 | 0.590862418 | |
| SERTAD1 | KEAT | 300 | 1.368468288 | 0.000723339 | *** |
| SERTAD1 | KEAT | 400 | 0.837081944 | 0.003135485 | ** |
| SERTAD1 | SHSY5Y | 100 | 0.488091407 | 0.975446513 | |
| SERTAD1 | SHSY5Y | 200 | 0.638547987 | 0.332692404 | |
| SERTAD1 | SHSY5Y | 300 | 1.303073933 | 0.000951148 | *** |
| SERTAD1 | SHSY5Y | 400 | 1.610861767 | 0.000987695 | *** |
| SERTAD1 | THP1 | 100 | 0.205696303 | 0.902448724 | |
| SERTAD1 | THP1 | 200 | 0.338764066 | 0.570029228 | |
| SERTAD1 | THP1 | 300 | 0.547079874 | 0.022872106 | * |
| SERTAD1 | THP1 | 400 | 0.764255217 | 0.017658179 | * |
| SLC7A11 | HEK293 | 100 | 0.149396047 | 0.849795497 | |
| SLC7A11 | HEK293 | 200 | 0.624007376 | 0.32095334 | |
| SLC7A11 | HEK293 | 300 | 1.024205487 | 0.0119543 | ** |
| SLC7A11 | HEK293 | 400 | 1.525681577 | 0.001839237 | ** |
| SLC7A11 | KEAT | 100 | 1.278422732 | 0.004857006 | ** |
| SLC7A11 | KEAT | 200 | 1.628777791 | 0.001279284 | ** |
| SLC7A11 | KEAT | 300 | 2.384745366 | 1.8371E−05 | *** |
| SLC7A11 | KEAT | 400 | 2.217042927 | 7.32868E−05 | *** |
| SLC7A11 | SHSY5Y | 100 | −0.103270227 | 0.981595517 | |
| SLC7A11 | SHSY5Y | 200 | 0.341538132 | 0.731722159 | |
| SLC7A11 | SHSY5Y | 300 | 0.892179043 | 0.007433299 | ** |
| SLC7A11 | SHSY5Y | 400 | 1.472451233 | 0.000846831 | *** |
| SLC7A11 | THP1 | 100 | 0.67493209 | 0.705482422 | |
| SLC7A11 | THP1 | 200 | 1.731317003 | 0.01494842 | * |
| SLC7A11 | THP1 | 300 | 2.829384823 | 3.10717E−05 | *** |
| SLC7A11 | THP1 | 400 | 3.604677326 | 1.52231E−06 | *** |
| TFE3 | HEK293 | 100 | 0.32559373 | 0.782459077 | |
| TFE3 | HEK293 | 200 | 0.388020175 | 0.361332002 | |
| TFE3 | HEK293 | 300 | 0.498372067 | 0.125432998 | |
| TFE3 | HEK293 | 400 | 0.619025387 | 0.03041186 | * |
| TFE3 | KEAT | 100 | 0.136322808 | 0.866912441 | |
| TFE3 | KEAT | 200 | 0.163443799 | 0.691407565 | |
| TFE3 | KEAT | 300 | 0.533933906 | 0.016387617 | * |
| TFE3 | KEAT | 400 | 0.515133821 | 0.040024315 | * |
| TFE3 | SHSY5Y | 100 | 0.181675527 | 0.975446513 | |
| TFE3 | SHSY5Y | 200 | 0.28982904 | 0.730424752 | |
| TFE3 | SHSY5Y | 300 | 0.343079912 | 0.12335345 | |
| TFE3 | SHSY5Y | 400 | 0.669577545 | 0.006021562 | ** |
| TFE3 | THP1 | 100 | 0.129197373 | 0.916945325 | |
| TFE3 | THP1 | 200 | 0.251414751 | 0.765102694 | |
| TFE3 | THP1 | 300 | 0.370635557 | 0.169707602 | |
| TFE3 | THP1 | 400 | 0.6015664 | 0.037856646 | * |
| TNFSF9 | HEK293 | 100 | 0.363851311 | 0.794827808 | |
| TNFSF9 | HEK293 | 200 | 0.371764512 | 0.504140993 | |
| TNFSF9 | HEK293 | 300 | 0.310229603 | 0.26407899 | |
| TNFSF9 | HEK293 | 400 | 0.773574355 | 0.028440055 | * |
| TNFSF9 | KEAT | 100 | 0.480754172 | 0.484855387 | |
| TNFSF9 | KEAT | 200 | 0.537395156 | 0.221087128 | |
| TNFSF9 | KEAT | 300 | 1.41958156 | 0.000298757 | *** |
| TNFSF9 | KEAT | 400 | 1.009012152 | 0.004118506 | ** |
| TNFSF9 | SHSY5Y | 100 | 0.270640033 | 0.975446513 | |
| TNFSF9 | SHSY5Y | 200 | 0.383682522 | 0.784963045 | |
| TNFSF9 | SHSY5Y | 300 | 0.644569749 | 0.034648225 | * |
| TNFSF9 | SHSY5Y | 400 | 1.009458058 | 0.006133312 | ** |
| TNFSF9 | THP1 | 100 | 0.289238635 | 0.898588673 | |
| TNFSF9 | THP1 | 200 | 0.586717315 | 0.522014401 | |
| TNFSF9 | THP1 | 300 | 0.806508733 | 0.011859845 | * |
| TNFSF9 | THP1 | 400 | 0.932370873 | 0.008480796 | ** |
| TXNRD1 | HEK293 | 100 | 0.224634327 | 0.794827808 | |
| TXNRD1 | HEK293 | 200 | 0.533236006 | 0.241293325 | |
| TXNRD1 | HEK293 | 300 | 0.573065825 | 0.089223218 | |
| TXNRD1 | HEK293 | 400 | 0.848432979 | 0.01532973 | * |
| TXNRD1 | KEAT | 100 | 1.251561616 | 0.004857006 | ** |
| TXNRD1 | KEAT | 200 | 1.498140354 | 0.001279284 | ** |
| TXNRD1 | KEAT | 300 | 1.960279219 | 1.85786E−05 | *** |
| TXNRD1 | KEAT | 400 | 2.004547767 | 6.25279E−05 | *** |
| TXNRD1 | SHSY5Y | 100 | 0.457156007 | 0.975446513 | |
| TXNRD1 | SHSY5Y | 200 | 0.944499615 | 0.034846485 | * |
| TXNRD1 | SHSY5Y | 300 | 1.145010781 | 0.000812452 | *** |
| TXNRD1 | SHSY5Y | 400 | 1.518334758 | 7.81531E−05 | *** |
| TXNRD1 | THP1 | 100 | 0.17539298 | 0.898588673 | |
| TXNRD1 | THP1 | 200 | 0.413715579 | 0.335216476 | |
| TXNRD1 | THP1 | 300 | 0.836424309 | 0.000878223 | *** |
| TXNRD1 | THP1 | 400 | 1.245949084 | 0.000104299 | *** |
| ZBTB21 | HEK293 | 100 | 0.128588902 | 0.853866141 | |
| ZBTB21 | HEK293 | 200 | 0.370361647 | 0.492022336 | |
| ZBTB21 | HEK293 | 300 | 0.335337008 | 0.212849163 | |
| ZBTB21 | HEK293 | 400 | 0.678269503 | 0.021506893 | * |
| ZBTB21 | KEAT | 100 | 0.455764909 | 0.3687932 | |
| ZBTB21 | KEAT | 200 | 0.564343964 | 0.120560066 | |
| ZBTB21 | KEAT | 300 | 0.999393779 | 0.000634969 | *** |
| ZBTB21 | KEAT | 400 | 0.860211826 | 0.00318253 | ** |
| ZBTB21 | SHSY5Y | 100 | 0.178247974 | 0.975446513 | |
| ZBTB21 | SHSY5Y | 200 | 0.513362154 | 0.324353261 | |
| ZBTB21 | SHSY5Y | 300 | 0.628695083 | 0.012884622 | * |
| ZBTB21 | SHSY5Y | 400 | 0.968459653 | 0.001463647 | ** |
| ZBTB21 | THP1 | 100 | −0.013339262 | 0.99268518 | |
| ZBTB21 | THP1 | 200 | 0.257647452 | 0.583902039 | |
| ZBTB21 | THP1 | 300 | 0.374585106 | 0.040509527 | * |
| ZBTB21 | THP1 | 400 | 0.586512132 | 0.005898475 | ** |
| ZFAND2A | HEK293 | 100 | 0.137596236 | 0.836902067 | |
| ZFAND2A | HEK293 | 200 | 0.337476578 | 0.494614416 | |
| ZFAND2A | HEK293 | 300 | 0.497914173 | 0.0946943 | |
| ZFAND2A | HEK293 | 400 | 0.701379158 | 0.038425812 | * |
| ZFAND2A | KEAT | 100 | 0.259188715 | 0.662030871 | |
| ZFAND2A | KEAT | 200 | 0.465830061 | 0.324377995 | |
| ZFAND2A | KEAT | 300 | 3.354085135 | 1.85786E−05 | *** |
| ZFAND2A | KEAT | 400 | 1.765825591 | 0.000199737 | *** |
| ZFAND2A | SHSY5Y | 100 | 0.091657898 | 0.979224069 | |
| ZFAND2A | SHSY5Y | 200 | 0.391882235 | 0.534343447 | |
| ZFAND2A | SHSY5Y | 300 | 0.532753735 | 0.034648225 | * |
| ZFAND2A | SHSY5Y | 400 | 1.065503232 | 0.003007589 | ** |
| ZFAND2A | THP1 | 100 | 0.125249017 | 0.933713424 | |
| ZFAND2A | THP1 | 200 | 0.2496621 | 0.760525755 | |
| ZFAND2A | THP1 | 300 | 0.567736282 | 0.053551705 | |
| ZFAND2A | THP1 | 400 | 1.235657138 | 0.003728618 | ** |

The transcriptomics data demonstrated a dose-dependent increase of various NRF2 target genes in four cell lines, including Heme Oxygenase 1 (HMOX1), Glutamate-cysteine ligase (GCLM, also known as gamma-glutamylcysteine synthetase), NAD(P)H Quinone Dehydrogenase 1 (NQO1), and Thioredoxin Reductase 1 (TXNRD1). Increased levels of HMOX1, GCLM, NQO1 and TXNRD1 mRNA and proteins were identified. The increased expression of target genes of NRF2 transcription factor at the protein level was confirmed by profiling proteins by untargeted mass spectrometry in PMA-differentiated THP-1 cell lysates after anatabine treatment (100, 200, 300, 400 μM) for 24 hours and observed intracellular protein products of NRF2 target genes, specifically HMOX1, GCLM, NQO1 and TXNRD1, all significantly expressed (adjusted p value<0.05).

Surprisingly, it was found that anatabine activates NRF2 as demonstrated by the increased NRF2 translocation shown in FIG. 3, in which the log2-fold change of NRF2 translocation was increased for 250 μM (P<0.01), 500 μM (P<0.001) and 1000 μM (P<0.001) of anatabine.

This NRF2 activation by anatabine is particularly surprising in view of the fact that similar tobacco alkaloids (e.g. nornicotine, nicotine, cotinine, anabasine) do not substantially affect the NRF2 pathway—see FIG. 4. Only anatabine provides a statistically significant NRF2 activation as shown by the significant increase of luciferase activity for anatabine at 250 μM (P<0.01), 500 μM (P<0.001) and 1000 μM (P<0.001). The log2-fold change for 1000 μM (P<0.001) is at least +7.

Cell viability for nornicotine and nicotine was below 85% at 250 μM, 500 μM, and 1000 μM and below 85% for cotinine and anabasine at 500 μM, and 1000 μM. In contrast, cell viability was above 85% for anatabine at 250 μM, 500 μM, and 1000 μM. A reduction in cell viability demonstrates that cells were dying at higher concentrations of nornicotine, nicotine, cotinine and anabasine but not at high concentrations of anatabine. Thus, anatabine provides significant NRF2 activation while at the same time being better-tolerated compared with similar tobacco alkaloids (e.g. nornicotine, nicotine, cotinine, anabasine).

Next, anatabine was compared with two known NRF2 activators; sulforaphane and dimethyl fumarate (DMF). DMF was analysed in two replicates from different providers (Alfa Aesar and Sigma). These data are shown in FIG. 5.

As expected, sulforaphane showed NRF2 activation as demonstrated by an increase in log2-fold change for 0.78 to 50 μM (P<0.01). The maximum log2-fold change for sulforaphane was approximately 5-fold at 12.5 μM.

Also as expected, DMF showed NRF2 activation in both replicates as demonstrated by an increase in log2-fold change for 125 μM and 250 μM (AlfaAesar: 125 μM, 6.74, P<0.01 and 250 μM, 6.81, P<0.01; and Sigma: 125 μM, 6.90, P<0.01 and 250 μM, 6.67, P<0.01). The maximum log2-fold change for DMF was approximately 6 to 7-fold at 125 μM and 250 μM.

For anatabine, the log2-fold change was significantly increased for 250 μM, 500 μM and 1000 with a maximum log2-fold change of over 7-fold at 1000 μM and approximately 3-fold at 500 μM (250 μM, 0.98, P<0.01; 500 μM, 2.92, P<0.01; 1000 μM, 7.3, P<0.01).

Moreover, anatabine appears to be better-tolerated than both sulforaphane and DMF. The cell viability was significantly reduced at higher concentrations of sulforaphane and DMF but not at higher concentrations of anatabine. In particular, the cell viability for 25 μM sulforaphane was around 50% and for 500 μM DMF was almost 0% in contrast to anatabine which still showed a cell viability of around 85% at 1000 μM. As mentioned above, a reduction in cell viability demonstrates that cells were dying. For this reason, higher concentrations of sulforaphane and DMF could not be tested but for anatabine a high cell viability % was observed throughout the tested concentration range, even at 1000 μM anatabine.

1.-12. (canceled)

13. A composition comprising anatabine or a salt thereof for treating a nuclear factor erythroid 2-related factor 2 (NRF2)-regulated disorder.

14. The composition according to claim 13, wherein the NRF2-regulated disorder is selected from one or more of a Herne Oxygenase 1 (HMOX1)-regulated disorder, a Glutamate-cysteine ligase (GCLM)-regulated disorder, a NAD(P)H Quinone Dehydrogenase 1 (NQO1)-regulated disorder, and/or a Thioredoxin Reductase 1 (TXNRD1)-regulated disorder.

15. The composition according to claim 14, wherein the HMOX1 and/or the GCLM is up-regulated.

16. The composition according to claim 14,
wherein the HMOX1-regulated disorder is Heme Oxygenase 1 Deficiency (HMOX1D) or chronic obstructive pulmonary disease (COPD), and/or
wherein the GCLM-regulated disorder is myocardial infarction.

17. The composition according to claim 13, wherein the NRF2-regulated disorder is a neurodegenerative disease, an inflammatory disease, an auto-immune disease, a cardiovascular disease, a respiratory disease, a digestive system disease, a neoplasm, and/or a glucose metabolism disease.

18. The composition according to claim 13, wherein the anatabine or salt thereof is a racemic mixture.

19. The composition according to claim 13, wherein the composition is administered daily.

20. The composition according to claim 13, wherein the composition is administered orally, topically, and/or by inhalation.

21. The composition according to claim 13, wherein the composition is administered as a daily dose comprising 0.1 mg to 400 mg, 0.5 mg to 350 mg, 1 mg to 300 mg, 2 mg to 250 mg, 3 mg to 200 mg, 4 mg to 150 mg, 5 mg to 100 mg, 6 mg to 90 mg, 7 mg to 80 mg, 8 mg to 70 mg, 9 mg to 60 mg, 10 mg to 50 mg, about 11 mg to about 45 mg, about 12 mg to 40 mg, 13 mg to 35 mg, 14 mg to 30 mg, 15 mg to 25 mg of anatabine or a salt thereof.

22. The composition according to claim 13, wherein the anatabine or the salt thereof is administered as a daily dose comprising 1 to 2 mg/kg.

23. The composition according to claim 13, wherein the anatabine or the salt thereof is administered as a daily dose comprising 1.6 mg/kg.

24. A composition comprising anatabine or a salt thereof for prophylactic treatment of a subject requiring NRF2 activation.

25. Anatabine or a salt thereof configured to be used as a NRF2 activator.
